(19) United States
(12) Patent Application Publication
(10) Pub. No.: US 2008/0313792 A1
(43) Pub. Date: Dec. 25, 2008

(54) Full-Face Motorcycle Goggles

(76) Inventor: Ernest J. Pino, San Jose, CA (US)

Correspondence Address: Clifton L. Anderson;Anderson Patents, P.O. Box 1210, Cupertino, CA, 95015, US

(21) Appl. No.: 11/766,073

(22) Filed: Jun. 20, 2007

(51) Int. Cl. A61F 9/02

(52) U.S. Cl. 2/427

(57) ABSTRACT

The present invention provides full-face polycarbonate goggles for use with an open-face helmet for comprehensive face and head protection for motorcycle riders. The goggles are optically transmissive so that, not only can the wearer see out, but also an onlooker can identify the wearer's facial features.

### BACKGROUND OF THE INVENTION

Motorcyclists require protection from the elements and accidents as they ride relatively exposed at high speeds. Many countries require motorcycle helmets to be worn to reduce the number of injuries and fatalities in motorcycle accidents. For the purposes herein, full-face helmets and open-face helmets can be distinguished.

A full-face helmet covers the entire head. Most full-face helmets have an open cutout with a plastic face shield (which may be clear or tinted) that generally swivels up and down to allow access to the eyes and nose. “Convertible” and “modular” variants feature a removable chin bar, while in “flip-up” and “flip-face” variants, the chin bar pivots upward. A removable or pivotable chin bar allows eating and drinking while the helmet is worn.

Despite offering the most protection, full-face helmets are often eschewed for many reasons, including their relative expense and bulk. These disadvantages can be doubled if a second helmet is to be carried for passengers, who do not always come with their own helmets. In addition, many riders dislike the increased heat, sense of isolation, lack of wind, and apparently reduced hearing associated with full-face helmets.

Accordingly, many riders prefer open-face helmets, including conventional open-face helmets and half helmets. Both conventional and half-helmet open-face variants lack the lower chin armor of the full-face helmet, as well as the face shield.

A conventional open-face helmet covers the back of the skull, and thus offers the same rear protection as a full-face helmet, while a half-helmet (i.e., a “shorty”) does not. Many open-face helmets offer visors of selectable length, some clear, some tinted, which may be used by the rider to block out sunlight or headlights.

By themselves, open-face helmets offer little protection to the face, even from non-crash events. Bugs, airborne particles such as dust and rain, or even wind can inflict discomfort and injury, as even small objects can be injurious at high speeds. Riders commonly wear goggles or at least wrap-around sunglasses to protect the eyes. In addition, riders often wear bandanas or leather masks during high-speed cruising to protect the portions of the face and mouth not protected by the goggles.

These measures for protecting the face are not entirely satisfactory. Bandanas and most leather masks do not offer much protection against pebbles that are often propelled reward of vehicles in front of a motorcycle. Form-fitting masks and to some extent bandanas can be plagued by moisture buildup around the mouth and nose. Also, bandanas and many masks leave skin around the goggles exposed to the elements. What is needed is improved protection for a rider's face when open-face and half helmets are used.

Herein, related art is described to facilitate understanding of the invention. Related art labeled “prior art” is admitted prior art; related art not labeled “prior art” is not admitted prior art.

### BRIEF DESCRIPTION OF THE DRAWINGS

FIG. 1 is a sequential perspective view of goggles in accordance with the present invention before and after being worn by a human.

FIG. 2 is a front view of the goggles of FIG. 1 on a human face.

FIG. 3 is a side view of the goggles of FIG. 1 on a human face.

FIG. 4 is a photograph showing a prototype of the goggles of FIG. 1 being worn by a human.

FIG. 5 is a photograph showing the prototype of FIG. 4 on another human. Yarn is attached to the prototype to provide wind speed and direction indications.

FIG. 6 is a flow chart of a method in accordance with the present invention.

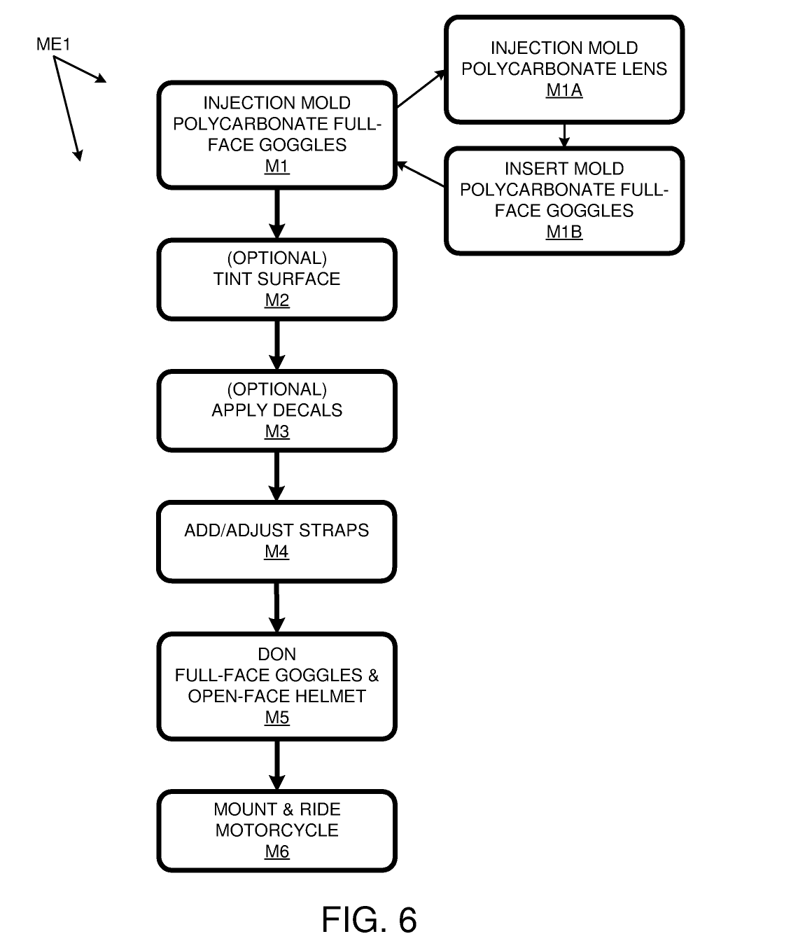

FIG. 7 is a perspective view of goggles in accordance with another embodiment of the invention.

### DETAILED DESCRIPTION

The present invention provides full-face goggles in which a monolithic structure of optically transmissive polymer, e.g., polycarbonate, is designed to extend at least from forehead to chin, temple to temple, and cheek to cheek to cover the eyes, nose, and mouth of the human wearer. Preferably, the goggles do not cover the crown of the head and the ears so as to accommodate an open-face helmet to be worn with the goggles.

The goggles can have vents that extend more orthogonally than along a “front” direction (a direction in which a nose extends from the rest of the face), to allow air intake while protecting the face from airborne particles and objects (including raindrops and other forms of precipitation) as the wearer face the direction of forward motorcycle motion. In case of an accident, the mask supplements the protection provided by an open-face helmet by protecting the facial area.

Although the basic material is transmissive, the invention allows surface treatments, e.g., for tinting or defining opaque designs (e.g., by applying decals). However, most embodiments of the invention leave sufficient area optically transmissive so that, not only can the wearer see out, but also so that others can recognize the wearer through the full-face goggles. The invention provides an effective two-piece (open-face helmet with full-face goggles) alternative to the one-piece (full-face helmet) and three-piece (open-face helmet, goggles, and mask) prior-art approaches to full-face protection for motorcycle riders.

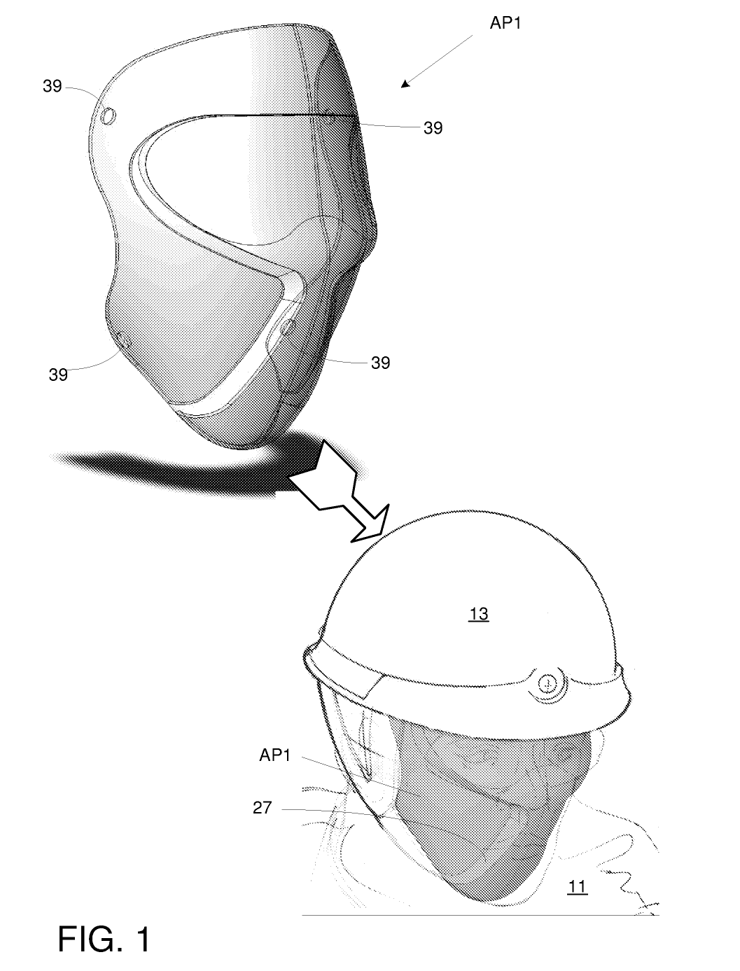

FIG. 1 depicts full-face goggles AP1 in accordance with the invention in isolation and worn by a rider 11 along with an open-face half helmet 13. The surface of the normally clear polycarbonate has been treated with a dye to provide a rose-colored tint to goggles AP1. Of course, other tints, e.g., amber and smoke-colored, can be applied instead. In this case, the tinting allows others to recognize the wearer through the goggles. Optionally, opaque areas, e.g., graphical figures and patterns, can be added, e.g., as decals, for decorative purposes as long as the rider's field of view is not obstructed.

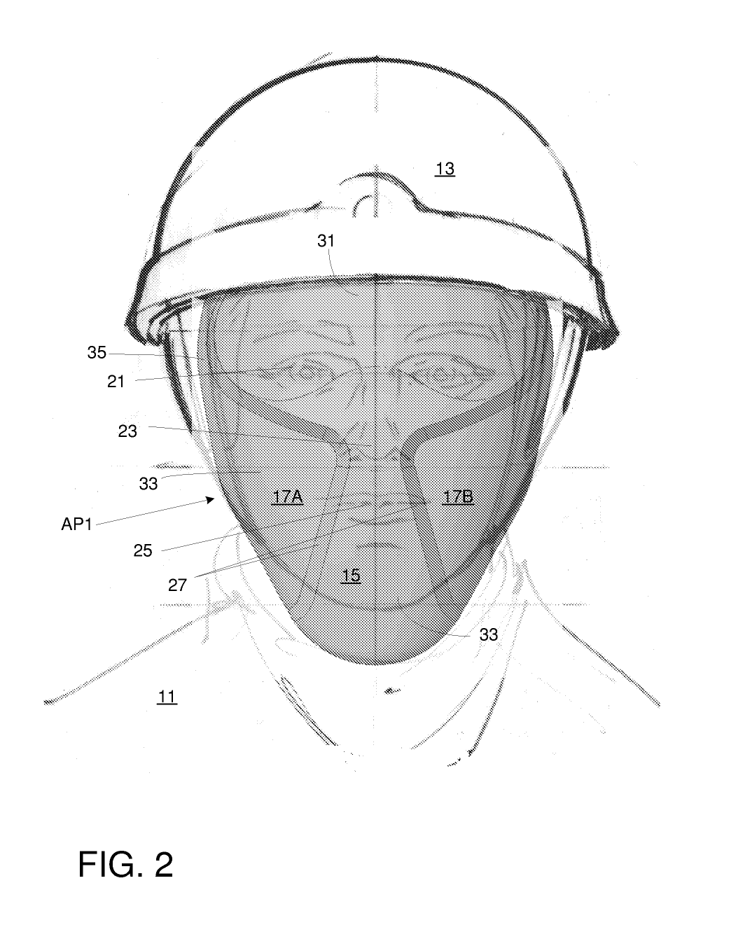

As best seen in FIG. 2, goggles AP1 include a horizontally centered raised area 15 between two recessed areas 17A and 17B. Raised area 15 extends over the rider's eyes 21, nose 23 and the middle of the mouth 25. Raised area 15 can accommodate eyewear, e.g., corrective glasses. Raised area 15 allows a greater volume of airflow to remove moisture from the mouth and nose areas and from the polycarbonate in front of the eyes. A forwardly extending bezel 27 spaces raised portion 15 from recessed portions 17A and 17B.

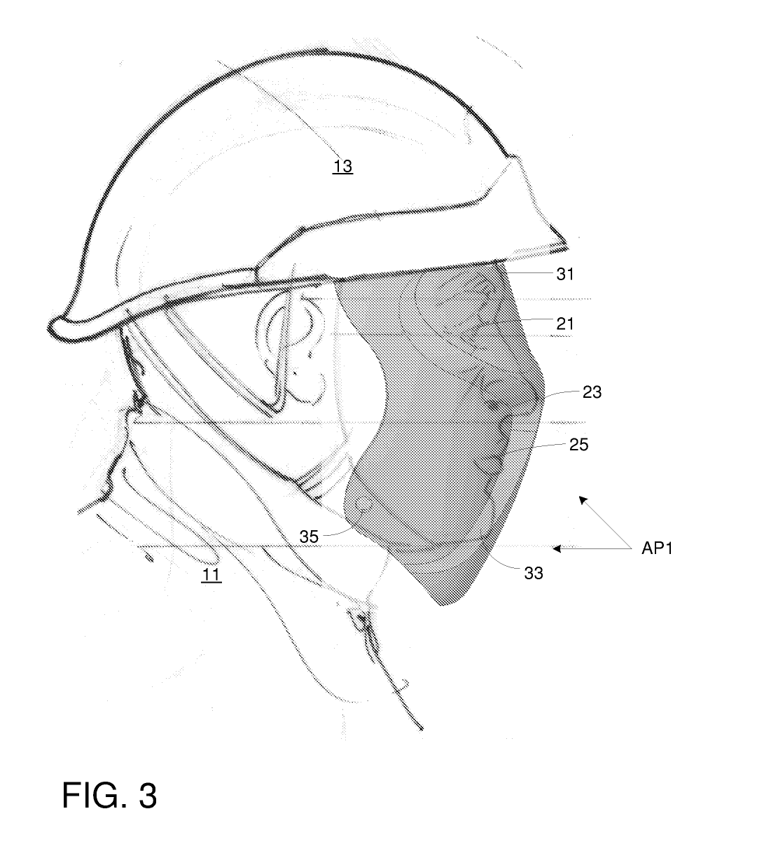

Goggles AP1 are sufficiently rigid that they hold their own shape, in contrast to a bandana or a leather mask, both of which rely on the underlying facial structure for their shape. As a result, goggles AP1 are spaced from face 29 over most of the goggle area, with contact being made only over a limited area, e.g., at the forehead 31 and chin 33. The exact area of contact depends on the shape of the wearer's face; in some cases, there is contact with the cheeks 35 and temples 37. Goggles AP1 provide sufficient space to provide ample airflow over nose 23 and mouth 25 for removing moisture, and to accommodate eyewear, such as corrective glasses, as best seen in FIG. 3. Goggles AP1 include apertures 39 for attaching straps (not shown), which, in turn, attach goggles AP1 to rider 11.

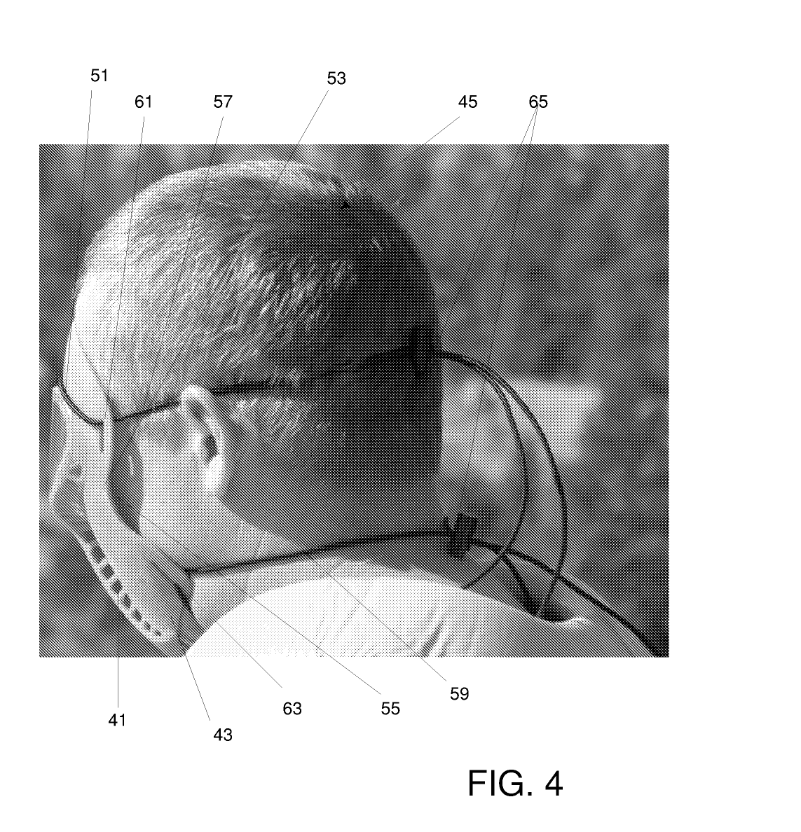

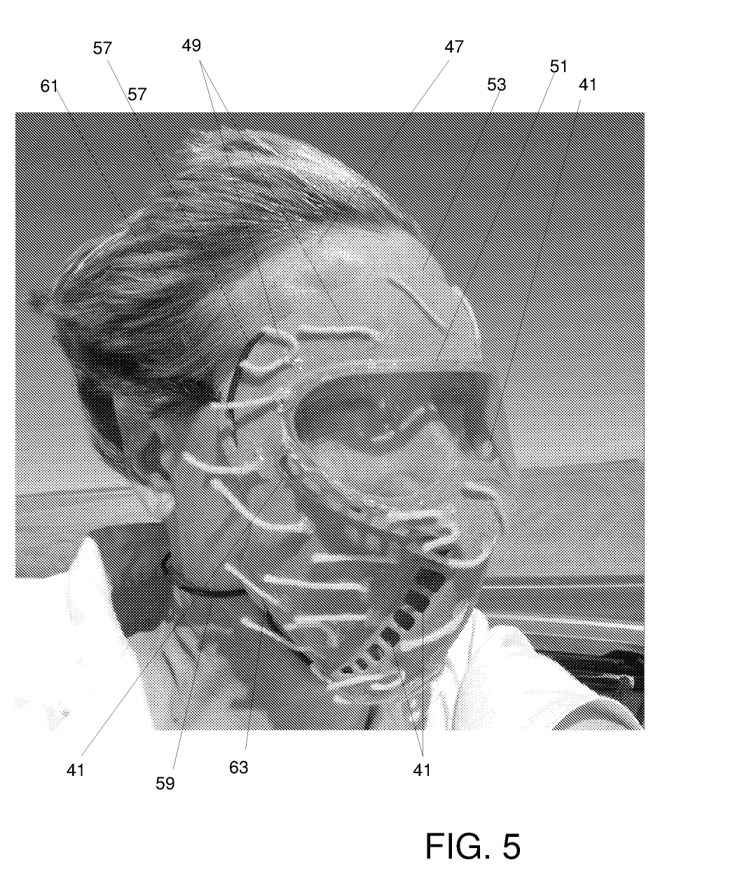

Although not shown in FIG. 1-3, orthogonal vents are provided along bezel 27. The vents are “orthogonal” in the sense that the directions of their respective depths are more orthogonal to than along the forward direction the rider is expected to be facing and moving while a motorcycle is in motion. Similar vents 41 are shown in the photographs of FIGS. 4 and 5, which depict a proof-of-concept prototype 43 of goggles AP1 on respective humans 45 and 47. (In FIG. 4, the subject is stationary, while in FIG. 5, the subject is riding a motorcycle with a speed of 60 mph. In FIG. 5, yarn 49 is glued to prototype 41 to serve as wind indicators.)

In addition to the vents visible in the views of FIGS. 4 and 5, proof-of-concept prototype 41 includes vents along the brow 51, i.e., the portion above the eyes, of bezel 27. The orthogonal orientation of the vents allows air to flow in, while insects, rain, dust, and other particles are deflected. Air taken in through vents 41 can exhaust about the perimeter 53 of prototype 43, e.g., over the wearer's cheeks through adjacent gaps 55 apparent in FIG. 4. The combination of vents and exhaust gaps allows airflow to remove moisture and to preserve the wind sensation desired by many motorcycle riders.

FIG. 5 and especially FIG. 4 show how goggles AP1 and prototype 43 can be secured to a head. Two straps 57 and 59 are attached to two respective pairs of apertures 61 and 63 in prototype 43. The strap lengths can be set and adjusted by moving respective clasps 65. One prototype has been modified by removing some material around an aperture so that a strap can be 25 slid off. This permits the mask to be removed without having to remove a helmet. Prototype 43 has a clear eye shield 65 glued to an opaque body 67. The preferred embodiments are monolithic molded pieces (excluding the straps) of optically transmissive material.

Goggles AP1 of FIGS. 1-3 are made by injection molding CR-39 polycarbonate at method segment M1 of method ME1 in accordance with the invention. The molding can involve molding the full structure at once or in steps using insert or over molding. Insert molding is an injection molding process whereby plastic is injected into a cavity and around an insert piece placed into the same cavity just prior to molding. The result is a single piece with the insert encapsulated by the plastic. For example, the lens, which requires higher optical quality, can be molded first at method subsegment M1A. Then the already formed lens can be insert molded (or over molded) with the rest of the full-face goggles at method subsegment M1B. Since the lens and rest of the goggles are of similar polycarbonate material, molecular bonding provides for both physical strength and leak resistance at the joint. This can provide a lens with higher optical quality while still providing a monolithic structure, e.g., so that the lens does not separate at high cruising speeds.

Optionally, the surface of the polycarbonate can be dyed to provide a tint at method segment M2. Also, decals or other surface treatments can be applied to provide custom designs and patterns at method segment M3. Straps 57 and 59 can be added and/or adjusted at method segment M4. A human 11 can don goggles AP1 and an open face helmet 13 at method segment M5. Finally, the human 11 can mount and ride a motorcycle while wearing goggles AP1 and helmet 13 at method segment M7.

Because of the relative rigidity and impact resistance of the preferred CR-39 material, goggles AP1 provide protection not only against insects, dust, and rain, but may protect against pebbles such as those sometimes rendered airborne by vehicles in front of a motorcycle. When goggles are not needed, it can be conveniently stored. In fact, two or more goggles can be stacked so they occupy little more than a single goggles. Furthermore, they can be stored inside a spare half or open-face helmet, so they add nothing to the actual volume required for storage. When conditions called for it, goggles can be donned to protect the rider from the elements and reduce injuries in case of an accident.

CR-39, allyl diglycol carbonate, is a plastic polymer commonly used in the manufacture of eyeglass lenses. CR 39 is a trade-marked product of PPG Industries. CR-39 is transparent in visible spectrum and almost completely opaque in the ultraviolet range. It has high abrasion resistance, in fact the highest abrasion/scratch resistance of any uncoated optical plastic. CR 39 is about half the weight of glass and index of refraction only slightly lower than that of crown glass, making it an advantageous material for eyeglasses and sunglasses lenses. A wide range of colors can be achieved by dyeing of the surface or the bulk of the material. CR-39 is also resistant to most of solvents and other chemicals, to gamma radiation, to aging, and to material fatigue.

Other embodiments employ alternative polycarbonate materials, including other polyaliphatic and polyaromatic carbonates. Some embodiments of the invention use optically transmissive materials other than polycarbonate, e.g., polymethyl methacrylate (PMMA). Hard coatings can be applied to improve scratch resistance.

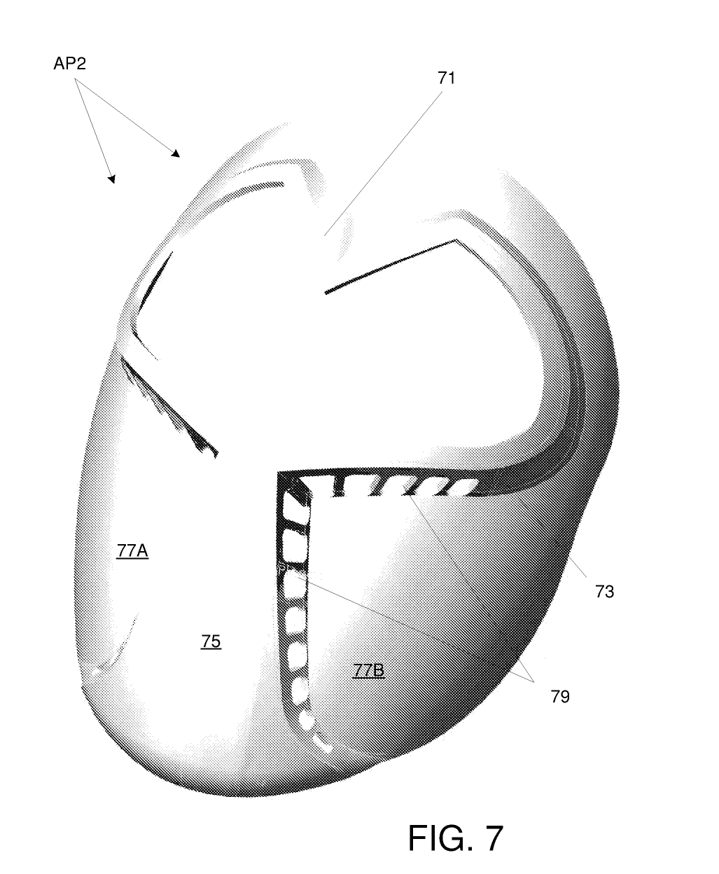

Since the inventive motorcycle goggles can be molded, e.g., injection molded, as a single piece, variants can be readily produced using different molds. For example, FIG. 7 shows goggles AP2 with a relatively angular design, particularly in a brow region 71, in accordance with the present invention. Like goggles AP1, goggles AP2 include a wall 73 spacing a raised area 75 from recessed areas 77A and 77B. Orthogonal vents 79 are formed in wall 73. “Orthogonal vents” is meant that the vents open (have depth) in a direction more orthogonal to than along a forward direction. In the illustrated embodiments, the vent depth dimensions are with 200 of an orthogonal to the forward direction.

By “optically transmissive” is meant transparent to visible light or sufficiently so that a human can identify facial features through the optically transmissive object. Optically transmissive is to be distinguished from translucent, which indicates that light can be transmitted through an object, but perhaps too diffusely to allow a human to identify facial features. By “rigid” is meant sufficiently rigid to hold its shape under its own weight. As used herein, “rigid” does not preclude some shape distortion under pressure. In the present context, “rigid” distinguishes over fabric masks (bandanas, leather masks), which tend to conform to the shape they are covering.

Clear goggles as well as (rose, smoke, amber, etc.) tinting can be produced. As indicated above, goggles can be personalized through painting and decals. The goggles can have grooves or other attachment means for attaching lens covers over (or behind) the lens area of the full-face googles for scratch protection and decoration.

Various approaches to attaching goggles can be used, including a Velcro top of the neck support, an over the ears strap, a lycra head sock attached to the goggles. It is also possible to mold in a mouth guard with the goggles; the mouth guard can then be used to retain the goggles. Although the contemplated application is for motorcycle riders, other applications (such as bicycle racing, snow skiing, water skiing, etc.) can make use of the invention. These and other variations upon and modifications to the detailed embodiments are provided for by the present invention, the scope of which is defined in the following claims.

What is claimed is:

1. Full-face goggles comprising:
a monolithic rigid structure of optically transmissive polymer configured to extend at least from forehead to chin and from cheek to cheek so as to cover the eyes, nose, and mouth of a human face, said polymer being a polycarbonate material or a polymethyl methacrylic material.

2. Full-face goggles as recited in claim 1 wherein said structure vent apertures with depths extending more orthogonal to than along a forward direction.

3. Full-face goggles as recited in claim 1 wherein said structure does not cover the crown or ears of a wearer.

4. Full-face goggles as recited in claim 1 wherein said polymer includes polycarbonate.

5. Full-face goggles as recited in claim 1 wherein said polycarbonate is CR-39 plastic.

6. Full-face goggles as recited in claim 1 wherein said goggles, while covering a wearer's eyes, nose, and mouth, do not cover said wearer's ears and crown.

7. A method of preparing for a motorcycle ride, said method comprising: donning an open-face helmet and full-face goggles including optically transmissive rigid polymer so that said goggles extend at least from forehead to chin and from cheek to cheek so as to cover the eyes, nose, and mouth of a human face, said plastic being a polycarbonate material or a polymethyl methacrylic material.

8. A method as recited in claim 7 further comprising mounting and ride a motorcycle.

9. A method as recited in claim 7 wherein said polymer is polycarbonate.

10. A method as recited in claim 9 wherein said polycarbonate is CR-39 plastic.

11. A method as recited in claim 7 further comprising injection molding said goggles.
